(19) United States
(12) Patent Application Publication
(10) Pub. No.: US 2026/0047645 A1
(43) Pub. Date: Feb. 19, 2026

(54) CONNECTION SYSTEM AND METHOD FOR RELEASING AN ADHESIVE CONNECTION

(71) Applicant: Gottlieb Binder GmbH & Co. KG, Holzgerlingen (DE)

(72) Inventors: Lars Heepe, Tübingen (DE); Dirk-Michael Drotlef, Tiefenbronn (DE)

(73) Assignee: Gottlieb Binder GmbH & Co. KG, Holzgerlingen (DE)

(21) Appl. No.: 19/294,555

(22) Filed: Aug. 8, 2025

(30) Foreign Application Priority Data
Aug. 13, 2024 (DE) 102024123073.8

(51) Int. Cl. A44B 18/00

(52) U.S. Cl. CPC A44B 18/0011; A44B 18/0015; A44B 18/0034

(57) ABSTRACT

A connection system has a first adhesive which has a plurality of adhesive elements. The first adhesive has a first free end and a connection region, wherein the connection region is arranged or formed on a carrier element preferably designed as a flexible tape. A first pivotable part of the first adhesive is movable, in particular, pivotable, relative to the carrier element. The first pivotable part has free edges. In a state in which the first pivotable part is not pivoted relative to the connection region, i.e., in which both lie in a common plane, the free edges in the plane are not directly surrounded by adhesive elements of the first adhesive. The first pivotable part has, in particular, a larger number of adhesive elements than the connection region. A method for releasing such a connection system is disclosed, wherein the connection system has a second adhesive.

### CROSS-REFERENCE TO RELATED APPLICATIONS

This application claims priority to German Patent Application No. DE 10 2024 123 073.8 filed on Aug. 13, 2024, the entire contents of which are hereby incorporated in full by this reference.

### DESCRIPTION

### Field of the Invention

The invention relates to a connection system with a first adhesive means, which has a plurality of adhesive elements, and a carrier element, wherein the first adhesive means has a first free end and is arranged or formed pivotably on the carrier element at a connection region spaced from the first free end, wherein a first pivotable part of the first adhesive means is movable relative to the carrier element and wherein the first pivotable part has a length L from the first free end of the first adhesive means to the beginning of the connection region. The invention also relates to a sanitary product and to a method for releasing an adhesive connection.

### Background of the Invention

Connection systems for the production of adhesive connections, in particular, manually releasable adhesive connections, often have flat adhesive means with a plurality of adhesive elements, which can be handled intuitively by a user-even a user of toddler age. When releasing such an adhesive connection, a free end of one of the adhesive means is usually grasped and separated from the other adhesive means by a peeling movement. The user will choose the peeling method intuitively, since in such case the force to be applied is largely determined by the width of a peel front. The amount of force to be applied is considerably less than if the user were to attempt to separate the adhesive means by means of a pushing movement or by lifting them vertically, wherein the force to be applied is largely determined by the surface area of the regions of the adhesive means that are adhered to one another. This means that the release of a corresponding adhesive connection is usually achieved by disposing a free end of one of the adhesive means or a carrier element connected to one of the adhesive means in the direction of the (still) adhered regions of the adhesive means.

The generic document EP 2 244 599 B1 describes a releasable fastening arrangement, for example in the form of a hook-and-loop fastener, wherein a flexible flap is formed within an adhesive means. The flexible flap, as well as the regions surrounding the flap, carries adhesive elements and can pivot out of the plane of the adhesive means. When the adhesive connection is separated by peeling, wherein a peel front moves in a direction from a free end of the flap to an attached end thereof, the flap initially remains attached while being passed by the peel front. Subsequently, a tensile force is applied to the flap due to the attached end, wherein the flap is not peeled but pulled off the corresponding adhesive means. The properties of a resulting peel resistance of the entire fastening arrangement, and thus of the force required to release a corresponding adhesive connection, can thus be adapted based on the design of the flap. In particular, the resulting peel resistance can be increased and/or discrete discontinuities of the peel resistance can be provided in certain regions.

U.S. Pat. No. 8,685,194 B2 describes a process for producing adhesive fasteners. In one embodiment variant, the method comprises forming discrete anti-peel flaps in an adhesive means by means of a plurality of slots. The flaps can be pivoted, for example, relative to a plane of a carrier material and are suitable for producing the above-described effect of converting a peeling principle into a pushing principle during a detachment process of the adhesive means from a corresponding counterpart. In some cases, the slots are formed on free edges of the adhesive means.

### SUMMARY OF THE INVENTION

### Object of the Invention

The object of the invention is to create a connection system which, by utilizing the known principle of the dependence of a force to be applied to release an adhesive connection on a peel angle or peel direction, offers advanced application options compared to the prior art. It is a further object of the invention to provide a sanitary product and a method for releasing an adhesive connection.

### DESCRIPTION OF THE INVENTION

This object is achieved by a connection system according to claim 1, a sanitary product according to claim 12, and a method according to claim 13. The dependent claims reflect preferred developments.

If a connection system of the type mentioned at the outset is in a state in which a first pivotable part of a first adhesive means is not angled relative to a connection region of the first adhesive means on a carrier element, free edges of the first pivotable part are not directly surrounded by adhesive elements of the first adhesive means.

In other words, this means that in relation to the free edges of the first pivotable part of the first adhesive means, even in a state in which the first adhesive means is not pivoted relative to a main plane of the planar carrier element and/or of a part of the first adhesive means attached to the carrier element, there are no further adhesive elements of the first adhesive means directly adjacent to both sides along the extension of the free edges in a main plane of the first pivotable part. Hence, there are no adhesive elements present at least on one side of a respective edge, within a distance that is less than or equal to the average distance between a plurality of the adhesive elements of the first adhesive means.

Preferably, the first pivotable part has an at least approximately rectangular shape, wherein three of the sides of the approximately rectangle form free edges, wherein the edge which is opposite the connection region simultaneously represents a first free end of the first adhesive means. However, a differently shaped first pivotable part is also conceivable. For example, the first free end can be rounded, which may result in a single, curved free edge, which, however, represents the free edges of the approximately rectangular arrangement over the course of its extension and is therefore considered in the present context as ‘multiple free edges with a smooth transition’. Furthermore, the first free end can be frayed or have multiple points, resulting in a plurality of free edges.

The maximum extension of a stretch from the first free end of the first adhesive means, in its longitudinal direction, to the beginning of the connection region determines a length L of the first pivotable part of the first adhesive means. The maximum extension of the first adhesive means perpendicular to length L determines its width B. Preferably, the carrier element has the same width or a greater width than the first adhesive means.

The first adhesive means comprises the plurality of adhesive elements, wherein at least one of the adhesive elements, but preferably several of the adhesive elements, is/are arranged or formed on the first pivotable part.

The carrier element is preferably designed as a flexible tape. In particular, the carrier element itself also has a free end. If the first adhesive means is attached to a second adhesive means, the release of the adhesive connection can be initiated by engaging the carrier element. A user will intuitively try to release the adhesive connection by peeling off the carrier element. However, in this peeling attempt, only the carrier element is initially moved until the connection region is reached. If peeling of the carrier element continues, a tensile force is applied to the first adhesive means after an imaginary peel front has reached the connection region. The force required to release the adhesive connection is considerably greater than that which would be necessary to actually peel off the first adhesive means.

To achieve actual peeling, a user must pull the free end of the carrier element against the intuitively chosen peel direction. The force applied is guided over a free length of the carrier element to the connection region, wherein a peel front starts there. The connection system has a safety feature due to the contrast between the intended method for separating the adhesive means and the intuitively obvious method. This prevents the adhesive connection from being released easily, in particular, by small children. Furthermore, the safety feature can be easily implemented during production.

The first pivotable part has, in particular, a larger number of adhesive elements than the connection region. Preferably, the first pivotable part comprises more than 50%, preferably more than 70%, particularly preferably more than 80% of the adhesive elements of the first adhesive means.

In a preferred embodiment of the connection system, the entirety of the adhesive elements of the first adhesive means is arranged or formed on the first pivotable part of the first adhesive means and/or on a second pivotable part of the first adhesive means. The second pivotable part is used in a development of the invention explained below. If the first adhesive means comprises the second pivotable part, the first and second pivotable part together preferably comprise more than 67%, particularly preferably more than 80% of the adhesive elements of the first adhesive means.

In order to avoid any additional resistance to a peel front starting at the transition between the connection region and the first pivotable part, it may be advantageous if the connection region is not provided with adhesive elements.

Preferably, the first pivotable part of the first adhesive means is a flat strip, wherein the ratio of length L to width B is in the range between 0.1:1 and 4:1, in particular, between 0.2:1 and 2:1.

The first adhesive means can be bonded to the carrier element, in particular, by means of a suitable adhesive or a thermal joining process.

The adhesive elements of the first adhesive means can, in particular, be hook elements, mushroom-shaped adhesive elements, hyperboloid-shaped adhesive elements or palm-shaped adhesive elements of a hook-and-loop or clip fastener. Optionally, the adhesive elements can be assigned to several of the aforementioned shapes. It is equally conceivable for the first adhesive means to comprise loops and/or fibers of a loop tape, velour tape, nonwoven fabric or a woven, knitted or crocheted fabric.

In a further embodiment variant, the first adhesive means can have nano- and/or microstructures. In particular, the first adhesive means can form projections with an area density of 10,000 projections to 100,000 projections per cm². Such area densities result in a strong adhesion force on a sufficiently smooth counter surface. The projections can have a height of at least 20 μm, of at least 30 μm, or of at least 40 μm. The projections generally have a height not greater than 800 μm, in particular, not greater than 150 μm. The aspect ratio of the projections, i.e., the projections' height to width ratio, is preferably between 0.1 and 3.0.

Preferably, the first adhesive means and/or the carrier element comprises plastic. Suitable materials include, for example, polypropylene, polyamide, polyethylene, polyurethane, polysiloxane and/or a thermoplastic elastomer. Particularly preferably, the carrier element and/or the first adhesive means comprises a bio-based and/or biodegradable plastic material. Suitable materials include, for example, polycaprolactone, polybutyrate, polybutylene succinate, cellulose acetate, bio-based polyethylene, polyethylene furanoate, bio-based polyamide, bio-based polyethylene terephthalate, bio-based polypropylene, polylactide, starch-based plastic and/or polyhydroxyalkanoate.

The maximum extension of a stretch from the free end of the carrier element, along its longitudinal direction, to the beginning of the connection region determines the free length T of the carrier element. Length T can preferably be up to 500% of length L of the first pivotable part of the first adhesive means. If length L is to be greater than length T, length L can alternatively be up to 500% of length T. Preferably, length T is in a range of ±20% of length L of the first pivotable part of the first adhesive means.

In a development of the invention, the first adhesive means has a second free end opposite the first free end, wherein the connection region is located between the first free end and the second free end. The first adhesive means comprises the mentioned second pivotable part. In the context of the development, a simple peeling off of the adhered first adhesive means along length L using the carrier element is no longer possible, since a peel front would have to run in two opposite directions from the connection region. In this way, an adhesive connection that is particularly difficult to release can be created within the framework of the aforementioned development. It can be advantageously released, in particular, by transverse stripping (along width B), although in this case a relatively high force is also necessary, since when pulling on the carrier element in the transverse direction the force application is not entirely suitable for forming a peel front.

The same effect can be achieved by arranging or forming a further first adhesive means on the carrier element, wherein the further first adhesive means is rotated by 180° in a plane of the carrier element with respect to the first adhesive means, wherein the connection region of the first adhesive means and a further connection region of the further first adhesive means are closer to one another than the respective first free ends of the two first adhesive means.

The length of the connection region and, if applicable, of the further connection region is preferably in a range of 0.1 mm to 10 mm. If the further connection region is present, the distance between the connection regions is preferably in the range of 0.1 mm to 100 mm.

The connection system preferably further comprises the second adhesive means, wherein the second adhesive means is suitable for producing an adhesive connection with the first adhesive means and/or the further first adhesive means. For this purpose, it can, in particular, comprise hook elements, mushroom-shaped adhesive elements, hyperboloid-shaped adhesive elements or palm-shaped adhesive elements, or fibers and/or loops of a loop tape, velour tape, nonwoven fabric or a woven, knitted or crocheted fabric. In the event that the first adhesive means and/or the further first adhesive means comprises nano- and/or microstructures, the second adhesive means can preferably have a surface that is sufficiently smooth to interact with the first adhesive means, for example in the form of a film.

The closure of a sanitary product, in particular, of a diaper, can be realized in a particularly advantageous manner by means of the connection system. One end of the carrier element opposite the free end of the carrier element is attached to the sanitary product. An arrangement of the carrier element on the sanitary product can preferably be realized by means of an adhesive or a thermal joining process. In particular, joining the sanitary product, the carrier element and the first adhesive means (and, if applicable, the further first adhesive means) can take place in one production step. It is also conceivable for the carrier element to be designed as an integral part of the sanitary product, wherein the attached end of the carrier element is marked by the transition of the main body of the sanitary product to the carrier element. Alternatively, the carrier element can be sewn to the main body of the sanitary product.

The carrier element, in conjunction with the first adhesive means (and possibly the further first adhesive means), forms a closure tab of the sanitary product. Preferably, the sanitary product further comprises the second adhesive means.

The closure tab can be arranged directly on a body of the sanitary product. In particular, the closure tab can be arranged directly on a diaper body of the diaper. Alternatively, the closure tab can be arranged on the body of the sanitary product via a further layer, in particular, in the form of a so-called ear, i.e., an ear-shaped layer. For example, the closure tab can be arranged on the diaper body via a diaper ear.

The object is further achieved by a method for releasing an adhesive connection of a connection system which comprises the first adhesive means (and optionally the further first adhesive means) and the second adhesive means. The method comprises disposing the free end of the carrier element in a direction from the attached end of the carrier element towards the free end of the carrier element, i.e., pulling the carrier element away from the attached end, so as to achieve peeling of the first adhesive means from the second adhesive means. This corresponds to the counterintuitive approach described above when releasing a generic adhesive connection. In particular, if peeling is completely prevented/made more difficult by a corresponding formation of the first adhesive means and/or of the further first adhesive means in the direction mentioned, the method provides for pulling the carrier element transversely to the direction mentioned. This separates the first adhesive means and/or, optionally, the further first adhesive means from the second adhesive means.

Further advantages of the invention can be found in the description and the drawings. Likewise, according to the invention, the aforementioned features and those which are to be explained below can each be used individually or together in any desired combinations. The embodiments shown and described are not to be understood as an exhaustive list, but, rather, have an exemplary character for the description of the invention.

### BRIEF DESCRIPTION OF THE DRAWINGS

In the drawings:

FIG. 1a shows a perspective view of a first adhesive means and a section of a carrier element;

FIG. 1b shows a further perspective view of the first adhesive means and a section of the carrier element of FIG. 1a;

FIG. 2a shows a section of a connection system comprising the first adhesive means, the carrier element and a second adhesive means, in a side view;

FIG. 2b shows a graph schematically illustrating the amount of force required to release the connection system of FIG. 2a, relative to a peel angle;

FIG. 3 shows a sectional side view of a further embodiment variant of a connection system;

FIG. 4 shows a sectional side view of a further embodiment variant of a connection system;

FIG. 5 shows a sectional side view of a further embodiment variant of a connection system;

FIG. 6a shows a perspective view of a further embodiment variant of a first adhesive means and of a section of a carrier element;

FIG. 6b shows a further perspective view of the first adhesive means and of the carrier element of FIG. 6a;

FIG. 6c shows a sectional side view of a connection system with the first adhesive means and the carrier element of FIGS. 6a and 6b and with a second adhesive means;

FIG. 7 shows a perspective view of a first adhesive means and of a further first adhesive means and a section of a carrier element;

FIG. 8 shows a sanitary product in the form of a diaper with a connection system;

FIG. 9 shows a diagram illustrating a method for releasing an adhesive connection.

### DETAILED DESCRIPTION OF EMBODIMENTS OF THE INVENTION

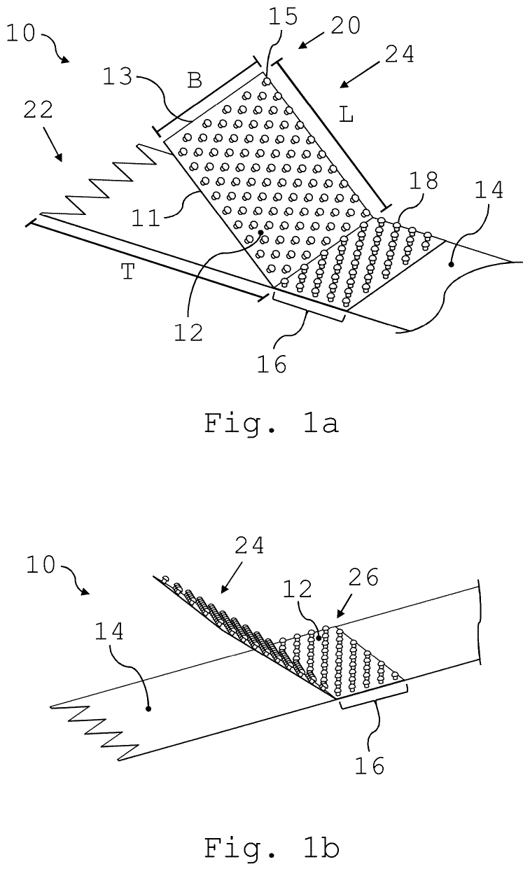

FIG. 1a shows a perspective view of a connection system 10 with a first adhesive means 12, which has three free edges 11, 13, and 15. The free edge 13 forms at the same time a first free end 20 of the first adhesive means 12. The first adhesive means 12 is arranged in a connection region 16 on a carrier element 14. A first pivotable part 24 of the first adhesive means 12 adjoins the connection region 16.

The first pivotable part 24 can be pivoted relative to the carrier element 14 and the connection region 16 and has a length L from the free edge 13 to the beginning of the connection region 16 and a width B from free edge 11 to free edge 15. The carrier element 14 is shown in section. It has a free end 22, wherein a length T defines the maximum length of the carrier element 14 from the free end 22 to the beginning of the connection region 16. In this case the length T is greater than the length L.

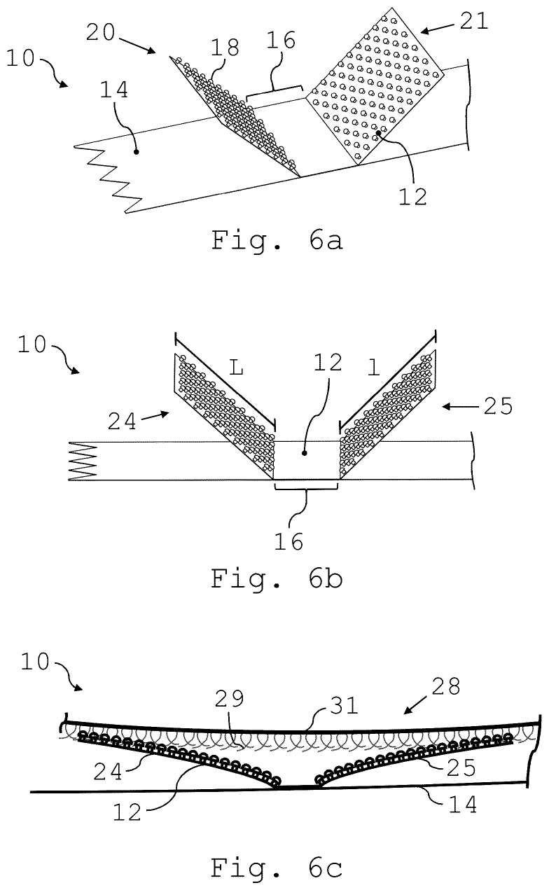

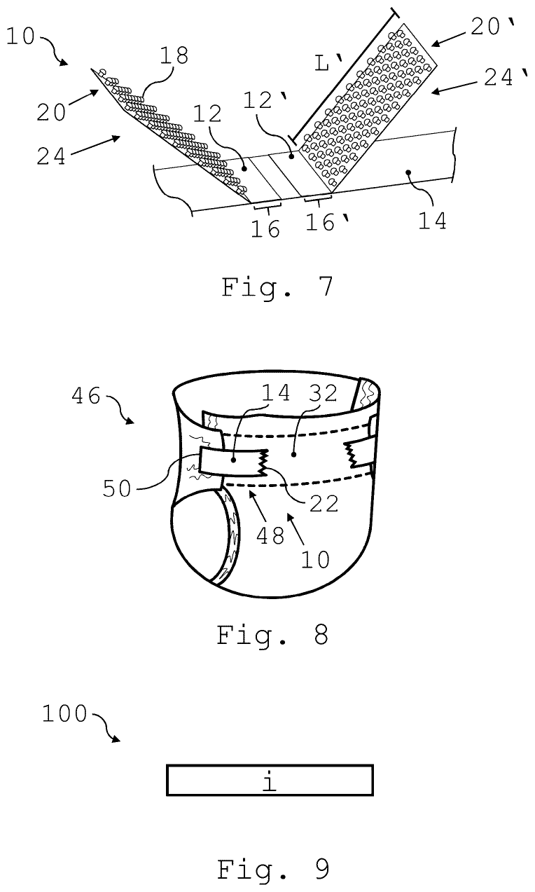

In the embodiment variant shown, the first adhesive means 12 carries a plurality of mushroom-shaped adhesive elements 18. The adhesive elements 18 occupy both the first pivotable part 24 and the connection region 16. The first pivotable part 24 has a larger number of mushroom-shaped adhesive elements 18 than the connection region 16. In the exemplary embodiment shown, approximately 70% of the existing adhesive elements 18 are located on the first pivotable part 24. In further embodiment variants (see FIGS. 6a and 7), the connection region 16 has no adhesive elements 18. It is also conceivable for the first pivotable part 24 to be formed on the carrier element 14 such that the connection region 16 is not perceivable as a separate region or as a separate layer on the carrier element 14, i.e., is part of the material of the carrier element 14 (not shown). Regions of the carrier element 14 that are not connected to the first adhesive means 12 can be pivoted equally relative to the first adhesive means 12 if the carrier element 14 is designed to be flexible.

FIG. 1b shows a further perspective view of the connection system 10 of FIG. 1a. The carrier element 14 is shown in section and the first pivotable part 24 of the first adhesive means 12 is pivoted relative to the carrier element 14 and the connection region 16. In the exemplary embodiment shown, the first adhesive means is arranged on the carrier element 14 by means of an adhesive 26.

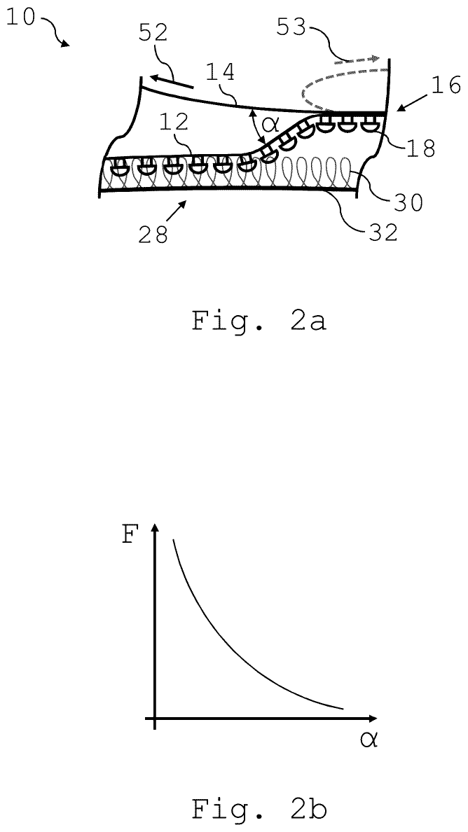

FIG. 2a shows a sectional side view of a further variant of a connection system 10, wherein the connection system 10 has a first adhesive means 12 and a carrier element 14, which are designed analogously to the embodiment in FIGS. 1a and 1b. The connection system 10 furthermore has a second adhesive means 28, here in the form of a loop tape 32. Loops 30 of the loop tape 32 are partially hooked with mushroom-shaped adhesive elements 18 of the first adhesive means 12. FIG. 2a schematically shows a state during detachment of the first adhesive means 12 from the second adhesive means 28. The carrier element 14 is held at its free end 22 (see FIG. 1a), pulled in the direction of the region still attached in the illustration and at the same time lifted off the second adhesive means 28 (which in the illustration corresponds to a direction of pull indicated by an arrow 52).

Once the connection region 16 has been separated from the second adhesive means 28, a peel front with a peel angle α is formed (see FIG. 2b). In a hypothetical separation attempt by intuitively peeling the carrier element 14 off (along a direction of pull 53), the first adhesive means 12 would not be peeled off the second adhesive means 28. Rather, the first adhesive means 12 would have to be peeled off the second adhesive means 28 evenly across the surface with significant force.

FIG. 2b shows in simplified form a relationship between the peel angle α in a range of 180° to 90° and a force F to be initially applied to detach the first adhesive means 12 (see FIG. 2a) from the second adhesive means 28 (see FIG. 2a). α is preferably between 160° and 90°.

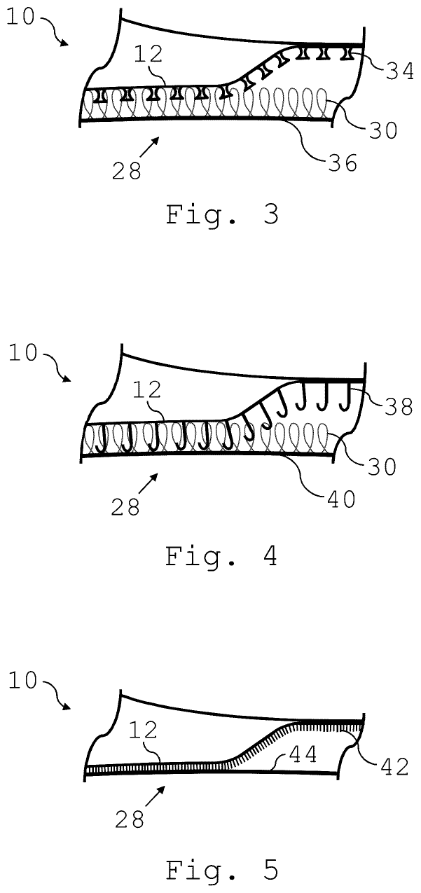

FIG. 3, FIG. 4 and FIG. 5 each show sectional views of further variants of a connection system 10. In analogy to FIG. 2a, the illustration includes a section of a first adhesive means 12 and of a second adhesive means 28, which are partially adhered to one another. The connection systems 10 differ in the type of adhesive means 12, 28 used. In FIG. 3, a first adhesive means 12 has palm-shaped adhesive elements 34 while a second adhesive means 28 has loops 30 of a woven fabric 36. In the exemplary embodiment shown, the palm-shaped adhesive elements 34 could equally be referred to as hyperboloid-shaped adhesive elements 34. In FIG. 4, a first adhesive element 12 has hook elements 38 for interaction with loops 30 of a velour tape 40. In FIG. 5, however, a first adhesive means 12 has nano- and/or microstructures 42 while a second adhesive means 28 has a smooth surface 44.

FIG. 6a shows a perspective view of a further variant of a connection system 10, wherein a first adhesive means 12 has a first free end 20 and a second free end 21. A carrier element 14 is again shown in section. A connection region 16 of the first adhesive means 12 is located between the first free end 20 and the second free end 21. In the embodiment variant shown, the connection region 16 does not carry any of the mushroom-shaped adhesive elements 18.

FIG. 6b shows a further perspective view of the connection system 10 of FIG. 6a. It becomes clear that the first adhesive means 12 comprises a first pivotable part 24 having a length L and a second pivotable part 25 having a length l. The lengths L and l can have the same value, as shown. However, this is not mandatory. The length of the connection region 16 can be selected depending on the application.

FIG. 6c shows a sectional side view of a connection system 10. The connection system 10 has the first adhesive means 12 shown in FIGS. 6a and 6b with the first pivotable part 24 and the second pivotable part 25 as well as the carrier element 14. In addition, the connection system 10 has a second adhesive means 28 which is partially adhered to the first adhesive means 12. The second adhesive means 28 has fibers 29 of a nonwoven fabric 31.

FIG. 7 shows a sectional perspective view of a further variant of a connection system 10. A first adhesive means 12 has a first free end 20, a connection region 16 and a first pivotable part 24. A further first adhesive means 12′ is arranged on a carrier element 14 such as to be rotated by 180° relative to the first adhesive means 12. In analogy to the first adhesive means 12, the further first adhesive means 12′ has a further first free end 20′, a further connection region 16′ and a further first pivotable part 24′. The connection system 10 shown in FIGS. 6a and 6b is reproduced in a functionally equivalent manner by means of the arrangement shown. As shown, one length L′ of the further first pivotable part 24′ can correspond to length L (see FIG. 1a) of the first pivotable part 24 or can differ therefrom. It is also conceivable for the first two adhesive means 12, 12′ to differ in terms of their width or with regard to the dimensions of their respective connection regions 16, 16′. It is also conceivable for the first adhesive means 12 and the further first adhesive means 12′ to each be provided with different adhesive elements 18 (cf. FIGS. 3, 4 and 5).

FIG. 8 shows a sanitary product 46, wherein in one of the presented variants a connection system 10 represents a closure tab 48 of the sanitary product 46. A first adhesive means 12 (not visible herein, see e.g., FIG. 1a) is adhered to a loop tape 32, which in turn is arranged or formed on the sanitary product 46. A carrier element 14 of the connection system 10 has an end 50 attached to the sanitary product 46 and a free end 22. Assuming that the connection system 10 is designed as shown in FIG. 2a, the closure tab 48 can be easily released by pulling on the free end 22 of the carrier element 14 in a direction from its attached end 50 towards the free end 22. However, if the free end 22 of the carrier element 14 is peeled off intuitively in the direction of the attached end 50, the closure tab 48 can be detached only with comparatively high force.

FIG. 9 shows a diagram of a method 100 for releasing a connection system 10 (see e.g., FIG. 8). Method step i involves pulling a carrier element 14 (see FIG. 8) in a direction away from an attached end 50 (see FIG. 8) of the carrier element 14 or, in particular, in a case where a second free end 21 (see FIG. 6a) and/or a further first free end 20′ (see FIG. 7) is present on a first adhesive means 12 (see e.g., FIG. 7), pulling the carrier element 14 transversely to a direction from the attached end 50 of the carrier element 14 to tv01he free end 22 of the carrier element 14. This results in the separation of the first adhesive means 12 and/or a further first adhesive means 12′ (see FIG. 7) from a second adhesive means 28 (see e.g., FIG. 2a).

As apparent from a combination of the figures of the drawing, the invention relates to a connection system 10 having a first adhesive means 12, which has a plurality of adhesive elements 18, 29, 30, 34, 38, 42. The first adhesive means 12 has a first free end 20 and a connection region 16, wherein the connection region 16 is arranged or formed on a carrier element 14 preferably designed as a flexible tape. A first pivotable part 24 of the first adhesive means 12 is movable, in particular, pivotable, relative to the carrier element 14. The first pivotable part 24 has free edges 11, 13, 15. In a state in which the first pivotable part 24 is not pivoted relative to the connection region 16, i.e., in which both lie in a common plane, the free edges 11, 13, 15 in said plane are not directly surrounded by adhesive elements 18, 29, 30, 34, 38, 42 of the first adhesive means 12. The first pivotable part 24 has, in particular, a larger number of adhesive elements 18, 29, 30, 34, 38, 42 than the connection region 16. The invention also relates to a method 100 for releasing such a connection system 10, wherein the connection system 10 has a second adhesive means 28.

### LIST OF REFERENCE SIGNS

10 Connection system
11 Free edge
12 First adhesive means
12′ Further first adhesive means
13 Free edge
14 Carrier element
15 Free edge
16 Connection region
16′ Further connection region
18 Mushroom-shaped adhesive element
20 First free end
20′ Further first free end
21 Second free end
22 Free end of the carrier element
24 First pivotable part
24 Further first pivotable part
25 Second pivotable part
26 Adhesive
28 Second adhesive means
29 Fiber
30 Loop
31 Nonwoven fabric
32 Loop tape
34 Palm-shaped/hyperboloid-shaped adhesive element
36 Woven fabric
38 Hook element
40 Velour tape
42 Microstructure
44 Smooth surface
46 Sanitary product
48 Closure tab
50 Attached end of the carrier element
52 Intended direction of pull
53 Intuitive direction of pull
100 Method for releasing a connection system
L Length of the first pivotable part
L′ Length of the further first pivotable part
l Length of the second pivotable part
B Width of the first adhesive means
T Free length of the carrier element
α Peel angle
F Force

What is claimed is:

1. A connection system, comprising:
a first adhesive means, which has a plurality of adhesive elements; and
a carrier element;
wherein the first adhesive means has a first free end and is arranged or formed pivotably on the carrier element at a connection region spaced from the first free end;
wherein a first pivotable part of the first adhesive means is movable relative to the carrier element and wherein the first pivotable part has a length L from the first free end of the first adhesive means to the beginning of the connection region;
wherein, in a state in which the first pivotable part is not angled relative to the connection region, free edges of the first pivotable part are not directly surrounded by the adhesive elements of the first adhesive means; and
wherein the first pivotable part has a greater number of adhesive elements than the connection region.

2. The connection system according to claim 1, wherein the first adhesive means has a second free end opposite the first free end, wherein the connection region is located between the first free end and the second free end, wherein the first adhesive means has a second pivotable part.

3. The connection system according to claim 1, wherein the ratio of length L of the first pivotable part of the first adhesive means and a width B of the first adhesive means, the extension of which is perpendicular to L, is between 0.7:1 and 4:1.

4. The connection system according to claim 1, wherein the first adhesive means is arranged in the connection region on the carrier element by means of an adhesive or by means of a thermal joining process.

5. The connection system according to claim 1, wherein the adhesive elements of the first adhesive means are:
a) hook elements; and/or
b) mushroom-shaped adhesive elements or palm-shaped adhesive elements or hyperboloid-shaped adhesive elements; and/or
c) fibers and/or loops of a loop tape or a velour tape or a woven fabric or a nonwoven fabric; and/or
d) nano- and/or microstructures for utilizing intermolecular forces.

6. The connection system according to claim 1, wherein the first adhesive means and/or the carrier element comprises plastic, being polypropylene, polyamide, polyethylene, polyurethane, polysiloxane and/or a thermoplastic elastomer.

7. The connection system according to claim 6, wherein the first adhesive means and/or the carrier element comprises a biodegradable plastic material and/or a bio-based plastic material, being polycaprolactone, polybutyrate, polybutylene succinate, cellulose acetate, bio-based polyethylene, bio-based polypropylene, polyethylene furanoate, bio-based polyamide, bio-based polyethylene terephthalate, polylactide, starch-based plastic and/or polyhydroxyalkanoate.

8. The connection system according to claim 1, wherein the carrier element has a free end, wherein a length T of the region of the carrier element from said free end to the beginning of the connection region is at most 500% of length L if T is chosen to be greater than L, or length L is at most 500% of length T if L is chosen to be greater than T.

9. The connection system according to claim 2, wherein the entirety of the adhesive elements of the first adhesive means is arranged or formed on the first pivotable part of the first adhesive means and/or on the second pivotable part of the first adhesive means.

10. The connection system according to claim 1, wherein a further first adhesive means is arranged or formed on the carrier element, wherein the further first adhesive means is rotated by 180° in a plane of the carrier element with respect to the first adhesive mean, wherein the connection region of the first adhesive means and a further connection region of the further first adhesive means are closer to one another than the first free end of the first adhesive means and a further first free end of the further first adhesive means.

11. The connection system according to claim 10, wherein the connection system has a second adhesive means for producing an adhesive connection with the first adhesive means and/or the further first adhesive means, wherein the second adhesive means has:
a) hook elements; and/or
b) mushroom-shaped adhesive elements or palm-shaped adhesive elements or hyperboloid-shaped adhesive elements; and/or
c) fibers and/or loops of a loop tape or a velour tape or a woven fabric or a nonwoven fabric; and/or
d) a sufficiently smooth surface for interaction with the micro- and/or nanostructures of the first adhesive means and/or the further first adhesive means.

12. A sanitary product, being a diaper, which has a connection system according to claim 1, wherein a closure tab of the sanitary product comprises the carrier element.

13. A method for releasing an adhesive connection with the connection system according to claim 11, comprising:
pulling the carrier element in a direction away from an attached end of the carrier element; or
pulling on the carrier element transversely to a direction from the attached end of the carrier element to the free end of the carrier element;
wherein separation of the first adhesive means and/or the further first adhesive means from the second adhesive means takes place.
